(19) United States
(12) Patent Application Publication
(10) Pub. No.: US 2026/0274781 A1
(43) Pub. Date: Sep. 17, 2026

(54) METHOD FOR RECOVERING HEAVY BY-PRODUCTS FROM THE MANUFACTURE OF ACRYLIC ACID

(71) Applicant: Arkema France, Colombes (FR)

(72) Inventors: Michel FAUCONET, Saint Avold Cedex (FR); Frederic SANDRE, Saint Avold Cedex (FR); Fanny DANTON, Saint Avold Cedex (FR)

(21) Appl. No.: 18/878,131

(22) PCT Filed: Jun. 20, 2023
(86) PCT No.: PCT/FR2023/050915

§ 371 Date: Dec. 23, 2024

(30) Foreign Application Priority Data
Jun. 24, 2022 (FR) FR22.06334

(51) Int. Cl. C07C 51/44

(52) U.S. Cl. CPC C07C 51/44

(57) ABSTRACT

A process for regenerating acrylic acid (AA), by thermal cracking, from heavy by-products (residues referred to as AAHP) from an AA production unit, with a view to recycling them in the acrylic acid production plant. This process includes two steps: hydrolysis and cracking, carried out batchwise in the same reactor, and improves the current performance of cracking plants.

### FIELD OF THE INVENTION

The present invention relates to a process for regenerating acrylic acid (AA), by thermal cracking, from heavy by-products (residues referred to as AAHP) from an AA production unit, with a view to recycling them in the acrylic acid production plant. This process consists of two steps: hydrolysis and cracking, carried out batchwise in the same reactor, and improves the current performance of cracking plants.

### TECHNICAL BACKGROUND

Under the effect of the temperature during the distillation steps, the manufacture of acrylic acid is accompanied by the formation of heavy compounds, derivatives of the addition of compounds having a nucleophilic property to the double bond of the unsaturated carbonyl-containing monomers, by the Michael reaction. Compounds having a boiling point higher than that of the acrylic monomer manufactured are referred to as “heavy” compounds.

In the case of an AA production unit, these are essentially:
derivatives of the addition of acrylic acid to the double bond of another acrylic acid molecule: 3-acryloyloxypropionic acid, also called “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers; and
derivatives of the addition of carboxylic acids, formed as by-products of the acrylic acid, or of water to the double bond of the AA or of the abovementioned oligomers.

The recovery of upgradable monomers from heavy Michael derivative compounds is difficult in the case of heavy products originating from an AA production unit. Specifically, during the thermal cracking process that regenerates acrylic acid, which is distilled and upgraded, a residue remains, the viscosity of which increases greatly when high cracking efficiencies are sought, until it can no longer be extracted from the cracking reactor.

The main factor limiting the efficiency of the regeneration of the compounds derived from the Michael reaction contained in the heavy streams from AA plants is the increase in the viscosity of the heavy residue obtained at the bottom of the cracker, when the fraction rich in acrylic monomers has been evaporated.

The evaporation of light compounds during the cracking results in a concentration of the heavy products in the residue stream and an increase in the viscosity of this stream. However, the residue has to remain sufficiently fluid after cooling to be transported and then treated for the purpose of destroying it.

In the case where there is production of light esters (methyl acrylate (MA) or ethyl acrylate (EA)) close to the AA production unit, a co-cracking of the respective heavy products can improve the situation, making the cracking residue more fluid. The proposed solution makes it possible to recover the maximum amount of AA per cracking operation while managing the viscosity of the residue formed without being dependent on another production unit.

Thus, in document EP 717 031, it has been shown that it is possible to improve the efficiency of the recovery of these upgradable noble products, if the cracking is carried out with a mixture of heavy products originating from an AA production unit and from an acrylic ester (EA) production unit, compared to the individual cracking of the heavy streams from these units. The effect of the addition of heavy products originating from the ester units (EAHP) to the heavy products originating from an AA unit (AAHP) is to reduce the viscosity of the final residue. The cracking reaction is carried out with mixtures having an AA heavy products/ester heavy products ratio of 9/1 to 1/9, at a temperature of 180° C. to 220° C., under atmospheric pressure, for a residence time of 0.5 to 3 hours. In this process, the cracking and the evaporation of the light compounds generated are carried out in a reactor, then the gas stream generated is sent into a distillation column and, finally, the bottom stream from the distillation column is recycled to the reactor. On the other hand, as the light fraction obtained by cracking consists mainly of AA and ester acrylic monomers, which are particularly sensitive to polymerization, the distillation stage necessarily has to be carried out under reduced pressure, so as to reduce the temperature, in order to prevent the formation of polymer in the column. Furthermore, the rectifying plates of the distillation column bring about the efficient separation of the polymerization inhibitors entrained in the gas mixture, which flow back to the column bottom, and consequently it is necessary to introduce fresh polymerization inhibitors at the column top, in order to prevent the formation of polymers in the upper part of the column.

For this reason, the reaction stage, carried out under higher pressure, and the distillation stage, carried out under reduced pressure, have to be separated. The installation for carrying out the process thus has to be equipped with a reactor and with a top condenser, which are operated at the same pressure, and with a distillation column operated at reduced pressure, fed with the condensed product, and comprising a boiler at the bottom and, at the top, a condenser, an item of reflux equipment and a feed of inhibitors. This arrangement is complicated and expensive.

Moreover, the co-cracking of AA heavy products mixed with EA heavy products leads to operating constraints. Specifically, when the ester unit is in shutdown, the cracking operation must be shut down. This leads to economic losses.

In other scenarios, the AA heavy products are thermally cracked batchwise without the addition of ester heavy products and generate an extremely viscous residue, which limits the performance of this cracking and causes problems in the storage and transfer of the residues.

To overcome the problem related to the viscosity, it is also known to add a solvent to the AA heavy products cracking residue.

Document EP 3255030 teaches the addition of higher alcohols during the cleavage of the residue, the maleic anhydride present in the residue being converted into maleic acid esters which are less sensitive to polymerization.

Document U.S. Pat. No. 6,414,183 teaches the dilution of the discharged residue with solvents such as acetic acid, water and methanol.

WO 2021/224044 describes a process for breaking down Michael adducts of acrylic acid, by dilution in a solvent I having a boiling point at 1013 hPa of at least 170° C. and a solubility in water at 25° C. of at least 20 g per 100 g of water, said solvent being chosen from alcohols such as ethylene glycol, propylene glycol, diethylene glycol, triethylene glycol and 2-ethoxyethanol, carboxamides such as N,N-dimethylacetamide, N-methylacetamide and N,N-dimethylformamide, sulfoxides such as dimethyl sulfoxide, and sulfones such as sulfolane.

However, this solution has several disadvantages, such as the generation of waste to be burned, if this is not an internal stream, or the provision of additional equipment for performing the mixing. Moreover, most of these solvents generate nitrogen-containing or sulfur-containing derivatives upon burning.

Consequently, there is a need to improve the regeneration efficiency, by thermal cracking, of heavy compounds originating solely from AA units.

### SUMMARY OF THE INVENTION

The invention relates to a process for regenerating a mixture of heavy by-products from an acrylic acid production unit (AAHP), said process comprising the following steps:
i. introducing said heavy by-products into a reactor;
ii. heating the heavy products at a temperature of between 80° C. and 200° C.;
iii. adding water simultaneously or at the final temperature until a water/AA heavy products ratio of 0.1 to 1.3 (limits included) is reached, progressively for a period of from 1 to 10 h, preferably from 1 to 5 h, resulting in a mixture of hydrolyzed products being obtained,
iv. in the same reactor, subjecting said mixture containing the hydrolyzed heavy products and water to batch thermal cracking, producing a gaseous overhead stream containing acrylic acid and water and a bottom stream (residue) concentrated in heavy products,
v. recovering a lighter fraction rich in AA and water that can be recycled to various points in the process,
vi. recovering said residue for the purpose of a removal treatment.

According to various implementations, said process comprises the following characteristics, if appropriate combined.

According to one embodiment, the pressure in the reactor is at atmospheric pressure or under slight pressure (pressure which can reach 2 MPa). According to one embodiment, the temperature at which the water is added varies between 80° C. and 200° C., preferably between 100° C. and 150° C.

According to one embodiment, the cracking temperature is between 140° C. and 260° C., preferably between 160° C. and 210° C.

According to one embodiment, the residence time of the reaction mixture in the cracking reactor is between 0.5 h and 10 h, preferably between 1 h and 4 h.

According to one embodiment, the thermal cracking reaction takes place at atmospheric pressure or under slight pressure (maximum 0.2 MPa).

According to one embodiment, said gaseous overhead stream containing acrylic acid and water is injected into a condenser.

According to one embodiment, the bottom stream from the reactor (residue) obtained on conclusion of the thermal cracking operation has a dynamic viscosity of less than 1 Pa·s, measured at a temperature of 100° C. for example using a Brookfield “CAP 1000+” viscometer of cone/plate type.

According to one embodiment, the thermal cracking reaction takes place in the absence of catalyst.

The present invention makes it possible to overcome the disadvantages of the prior art. It makes it possible to recover the maximum amount of AA per cracking operation, while managing the viscosity of the residue formed without being dependent on another production unit. This is accomplished by combining a step of hydrolysis pretreatment of the heavy by-products from an acrylic acid production unit with continuous addition of water, followed by a step of batch thermal cracking of the hydrolyzed products, the two steps being carried out in the same reactor.

The main advantages of the process according to the invention are:
a simple process that is inexpensive in terms of investment since in practice it requires no additional equipment compared to the cracking step alone;
a process which makes it possible to reduce the discharges by decreasing the amount of cracking residue;
a lower cracking temperature compared to cracking without a hydrolysis step, therefore enabling energy savings;
a less viscous residue compared to cracking without a hydrolysis step;
the cracking step does not depend on other units (especially ester units);
the hydrolysis also makes it possible to avoid a special treatment of the residues in order to ensure the discharging thereof, such as the addition of solvent for example in order to fluidify them;
an increase in recovery efficiency of noble products from the heavy Michael derivative products present in the streams of heavy products from AA plants, by pushing the cracking reaction further, the viscosity limit of the residue being reached later compared to a solution without hydrolysis.

### FIGURES

FIG. 1 diagrammatically represents an embodiment of an installation according to the invention.

### DESCRIPTION OF EMBODIMENTS OF THE INVENTION

The invention is now described in greater detail and in a nonlimiting manner in the description that follows.

The term “heavy by-products originating from a unit for the production of acrylic acid” comprises:
derivatives of the addition of acrylic acid to the double bond of another acrylic acid molecule: 3-acryloyloxypropionic acid, also called “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers;
derivatives of the addition of carboxylic acid formed as by-products of acrylic acid (for example acetic acid) or of water to the double bond of the AA or of the abovementioned oligomers.

The invention is based on a batch thermal cracking process, coupled with a batch hydrolysis operation carried out beforehand on the heavy by-products from an AA production unit, the two steps taking place in the same reactor. This may be a conventional stirred type reactor, or a heat exchanger.

The acrylic monomers involved in the Michael addition derivatives can be regenerated by hydrolyzing the oligomers prior to the heat treatment step. This hydrolysis reaction forms hydroxypropionic acid (HPA), which can be thermally cracked to give acrylic acid. Hydrolysis makes it possible to reduce the oligomer chains, making the residue less viscous.

Hydrolysis in batch mode is carried out under a pressure ranging from 0.1 to 2 MPa.

The regeneration efficiency (expressed as cracking efficiency) depends essentially on:
a/parameters for the hydrolysis: temperature and pressure, hydrolysis residence time and water/AA heavy products ratio, and
b/parameters for the cracking: temperature and residence time of the heat treatment.

The increase in these last two parameters (b/) tends to improve the regeneration efficiency, but this takes place at the expense of an increase in the viscosity of the cracking residue.

Cracking performance is characterized by two values:
the URR or useful recovery rate: this is the amount of acrylic acid recovered after cracking relative to the amount of AA heavy products which feed the cracker:

$$URR = mass of AA recovered / AA heavy products in cracker feed$$

the cracking rate or efficiency: this is the amount of acrylic acid recovered after cracking relative to the sum of the upgradable compounds in the cracker feed (acrylic acid (AA), acrylic acid dimers (AA2) and hydroxypropionic acid (HPA)).

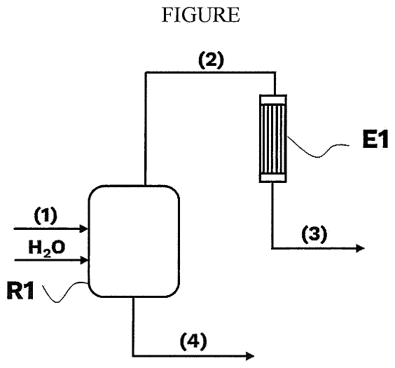

According to the embodiment of the process shown in FIG. 1, the stream from the acrylic acid production plant (AAHP) is introduced into the reactor R. The contents of the reactor are heated to the required temperature. The water is added progressively, simultaneously with the heating or at the final temperature, for a defined period in order to reach the desired water/AA heavy products ratio. Once the addition is complete, the reactor is heated to the temperature required to crack the Michael addition derivatives to give lighter compounds which are extracted in the form of a gas mixture (2) at the top of the reactor.

This vapor stream rich in acrylic acid and containing some heavy compounds including inhibitors at a low concentration, is advantageously recycled to the acrylic acid production process, either directly in vapor form, or after total condensation in a condenser E1 as stream (3).

According to one embodiment, at least one polymerization inhibitor is introduced into the condenser E1. These inhibitors are chosen from polymerization inhibitors known to a person skilled in the art: phenol derivatives, such as hydroquinone and its derivatives, for instance hydroquinone methyl ether, 2,6-di(tert-butyl)-4-methylphenol (BHT) and 2,4-dimethyl-6-(tert-butyl) phenol (Topanol A), phenothiazine and its derivatives, manganese salts, such as manganese acetate, salts of thiocarbamic or dithiocarbamic acid, such as metal thiocarbamates and dithiocarbamates, for instance copper di(n-butyl)dithiocarbamate, N-oxyl compounds, such as 4-hydroxy-2,2,6,6-tetramethylpiperidineoxyl (4-OH-TEMPO), compounds having nitroso groups, for instance N-nitrosophenylhydroxylamine and its ammonium salts, amine compounds, such as para-phenylenediamine derivatives, or a mixture of these inhibitors.

The residue stream recovered at the reactor bottom (4) is cooled and then removed in the form of a liquid of moderate viscosity, so as to be able to be transported without difficulty by pump, for example as far as a storage tank or an incineration unit.

### EXAMPLES

The following examples illustrate the invention without limiting it.

The depletion rate is defined by the mass of distillate/mass of heavy products ratio. In the case of addition of water, this rate becomes the “corrected depletion rate” by subtracting this mass of water from the amount of distillate.

### Example 1: Batch Hydrolysis and Cracking in the Same Reactor (According to the Invention)

The assembly used for the cracking operation consists of a 500 ml jacketed glass reactor equipped with a stirrer, a temperature probe immersed in the liquid phase, a vertical pipe in the upper part, for the extraction of the vapors, and a condenser. The AA heavy products are introduced into the reactor and then heated to the desired temperature. The water is added at this temperature over a well-defined period in an amount according to the chosen water/AAHP ratio. When the addition of water is complete, the mixture is heated at the cracking temperature until the targeted depletion rate is reached. The liquid (distillate) is collected in a receiving flask and analyzed. The residue is discharged through the bottom valve of the reactor.
Duration of the test: 7 h
Hydrolysis: T=146° C., residence time: 6 h, atmospheric pressure, water/AA heavy products ratio=0.5
Cracking: T=178° C., atmospheric pressure, residence time: 1 h, water/AA heavy products ratio=0.5
   URR=35.30%
   Cracking rate: 52%
   Corrected depletion rate=31.4%
   Viscosity=0.085 Pa·s

### Example 2 (Comparative): Cracking without Prior Hydrolysis

In this case, a known mass of AA heavy products is directly introduced into a glass reactor with a total volume of 500 cm³, heated by recirculating oil through its double wall.

The reactor is equipped with a stirrer, a temperature probe immersed in the liquid phase, a vertical pipe in the upper part, for the extraction of the vapors, and a condenser. The liquid (distillate) is collected in a receiving flask and analyzed.

Heating for 6 h, without addition of water, at 146° C., then temperature increase: Tmax=183° C., atmospheric pressure, residence time: 1 h, water/AA heavy products ratio=0.5.
URR=3.0%
Viscosity=0.068 Pa·s
Depletion rate=3.5%
Cracking rate=4.5%

This example makes it possible to show that at 183° C. there is little or no cracking.

### Example 3 (Comparative): Cracking without Prior Hydrolysis

Same protocol as for example 2. Heating for 6 h, without addition of water, at 146° C., then temperature increase: Tmax=199° C., atmospheric pressure, residence time: 1 h, water/AA heavy products ratio=0.5.
URR=29.2%
Viscosity=0.41 Pa·s
Depletion rate=32.7%
Cracking rate=44%

The comparative examples make it possible to show that implementation of the cracking after a hydrolysis step can be performed at a temperature lower than that required for the cracking of a stream that has not undergone the hydrolysis step. Specifically, in example 2, the maximum temperature is 183° C. and the cracking rate is equal to 4.5% versus 52% for example 1 where the maximum temperature is 178° C. Increasing the cracking temperature to 199° C. made it possible to obtain a cracking rate of 44%. However, the viscosity of the residue is 5 times higher.

1. A process for regenerating heavy by-products from an acrylic acid production unit (AAHP), said process comprising the following steps:
i. introducing said heavy by-products into a reactor;
ii. heating the heavy by-products at a hydrolysis temperature of between 80° C. and 200° C.;
iii. adding water simultaneously or at a final temperature until a water/acrylic acid (AA) heavy products ratio of 0.1 to 1.3 is reached, progressively for a period of from 1 to 10 h to obtain a mixture of hydrolyzed products;
iv. in the same reactor, subjecting said mixture containing the hydrolyzed heavy products and water to batch thermal cracking, producing a gaseous overhead stream containing acrylic acid (AA) and water and a bottom stream comprising residue concentrated in heavy products,
v. recovering a lighter fraction rich in AA and water that can be recycled to various points in the process,
vi. recovering said residue for the purpose of a removal treatment.

2. The process as claimed in claim 1, wherein step i) is carried out at atmospheric pressure or at a pressure which can range up to 2 MPa.

3. The process as claimed in claim 1, wherein the hydrolysis temperature is between 100° C. and 150° C.

4. The process as claimed in claim 1, wherein a cracking temperature is between 140° C. and 260° C.

5. The process as claimed in claim 1, wherein a residence time of the reaction mixture in the cracking reactor is between 0.5 h and 10 h.

6. The process as claimed in claim 1, in which the bottom stream obtained on conclusion of the batch thermal cracking has a dynamic viscosity, measured at 100° C., of less than 1 Pa·s.

7. The process as claimed in claim 1, further comprising a step of injecting said gaseous overhead stream containing acrylic acid and water into a condenser.

8. The process as claimed in claim 7, wherein at least one polymerization inhibitor is introduced at said condenser.

9. The process as claimed in claim 1, wherein the thermal cracking takes place in the absence of catalyst.

10. The process as claimed in claim 1, wherein step iii. comprises adding water for a period of from 1 to 5 h.

11. The process as claimed in claim 4, wherein the cracking temperature is between 160° C. and 210° C.

12. The process as claimed in claim 5, wherein the residence time of the reaction mixture in the cracking reactor is between 1 h and 4 h.
